(19) United States
(12) Patent Application Publication
(10) Pub. No.: US 2026/0054032 A1
(43) Pub. Date: Feb. 26, 2026

(54) METHOD FOR PRODUCING A CATHETER BODY

(71) Applicants: Heraeus Medevio GmbH & Co. KG, Hanau (DE); Contract Medical International GmbH, Dresden (DE)

(72) Inventors: Ilias NIKOLAIDIS, Hanau (DE); Mario HEINTZE, Dresden (DE); Llewellyn Wallace GROENEVELD, Dresden (DE); Raffael KUNZ, Dresden (DE)

(21) Appl. No.: 19/302,574

(22) Filed: Aug. 18, 2025

(30) Foreign Application Priority Data
Aug. 21, 2024 (DE) 102024123841.0

(51) Int. Cl. A61M 25/00

(52) U.S. Cl. CPC A61M 25/0012; A61M 2205/0233; A61M 2205/0238

(57) ABSTRACT

The present invention relates to a method for producing a catheter, comprising the method steps of providing a catheter body, creating at least one contact opening in the vicinity of the distal catheter body end, wherein, to create the contact opening, parts of the outer insulation and of any insulating layers of the flexible circuit board are removed so that the at least one electrical conductor can be selectively electrically contacted via the contact opening; providing a contact means; applying the contact means into the at least one contact opening; providing at least one electrode; positioning the electrode at the position of the contact opening in contact with the contact means in the contact opening; and, targeted heating of the electrode and of the contact means in order to secure the electrode to the electrical conductor via the contact means in an electrically conductive manner.

### CROSS-REFERENCE TO RELATED APPLICATION

This application claims priority pursuant to 35 U.S. C. 119(a) to German Patent Office Application No. 102024123841.0, filed Aug. 21, 2024, which application is incorporated herein by reference in its entirety.

### FIELD

The present invention relates to a method for producing a catheter, comprising the method steps of:
a) providing a catheter body, comprising
   a hollow cylindrical inner liner having an inner liner outer surface, the inner liner extending in a longitudinal direction from a proximal catheter body end to a distal catheter body end of the catheter body,
   an outer insulation having an inner insulation surface, the outer insulation coaxially surrounding the inner liner,
   a flexible circuit board which comprises at least one electrical conductor, wherein the flexible circuit board is arranged between the inner liner outer surface and the insulation inner surface, wherein the flexible circuit board is wound at least in sections in a spiral manner in turns around the inner liner and wherein the inner liner and the outer insulation are connected to one another, in particular integrally, in the spaces between the turns of the flexible circuit board;
b) creating at least one contact opening in the vicinity of the distal catheter body end, wherein, to create the contact opening, parts of the outer insulation and of any insulating layers of the flexible circuit board are removed so that the at least one electrical conductor can be selectively electrically contacted via the contact opening;
c) providing a contact means;
d) applying the contact means into the at least one contact opening;
e) providing at least one electrode;
f) positioning the electrode at the position of the contact opening in contact with the contact means in the contact opening;
g) targeted heating of the electrode and of the contact means in order to secure the electrode to the electrical conductor via the contact means in an electrically conductive manner.

### BACKGROUND

Catheters have been used in medical procedures for many years. Among other things, catheters can be used in medical procedures to examine, diagnose and/or treat tissue while being used in areas of the body, such as blood vessels, that are not easily accessible without more invasive procedures. For example, catheters can be used to deliver electrical energy to selected locations in the human body, such as the heart, to specifically kill tissue, such as heart tissue. This is often referred to as “ablation”. Catheters can also be used to stimulate body tissue. During stimulation, the amount of energy transferred to the body tissue is usually lower than during ablation.

Another procedure, often called “mapping,” uses a catheter with sensing electrodes to monitor various forms of bioelectrical activity in the human body.

Catheters can therefore be used both to transmit (during “ablation” and “stimulation”) and to absorb (during “mapping”) energy to or from body tissue.

Regardless of the direction of energy flow, catheters often contain a plurality of electrodes, i.e., two or more, at a distal catheter body end region, wherein the distal catheter body end region often describes the end that is inserted into the patient. Typically, the catheter contains an equal number of electrodes at its proximal catheter body end region, which are in electrical contact with the electrodes at the distal catheter body end region. The electrodes at the proximal catheter body end region are used to connect electrical devices according to the desired areas of application of the catheter.

The production of such catheter bodies with many electrodes is complex. Typically, the electrodes are electrically connected along the length of the catheter body preferably using electrically insulated wires. These wires take up space that could be used for other things (e.g., guide wires), require a larger diameter of the catheter body, are difficult to handle and time-consuming to assemble. This can be remedied by using flexible circuit boards instead of individual wires, as described, for example, in WO 2014/066470 A1. However, the electrical connection of flexible circuit boards to the catheter electrodes is difficult when using said boards, due to the comparatively low mechanical and, above all, thermal stability of the other components of the catheter body. Heating of other components can lead to undesirable deformations, which can negatively affect the functionality of the catheter.

One object of the present invention is to overcome, at least in part, one or more of the disadvantages resulting from prior art.

It is a further object of the invention to provide a method for producing a catheter body which is as simple, reliable in use and cost-effective as possible and, in particular, has the lowest possible thermal stress on the components of the catheter body during the electrically conductive securing of electrodes to a flexible circuit board.

A contribution to the at least partial fulfillment of at least one of the aforementioned objects is made by the features of the independent claims. The dependent claims provide preferred embodiments that contribute to the at least partial fulfillment of at least one of the objects.

A first embodiment of the invention is a method for producing a catheter, comprising the method steps of:
a) providing a catheter body, comprising
   a hollow cylindrical inner liner having an inner liner outer surface, the inner liner extending in a longitudinal direction from a proximal catheter body end to a distal catheter body end of the catheter body,
   an outer insulation having an inner insulation surface, the insulation coaxially surrounding the inner liner,
   a flexible circuit board which comprises at least one electrical conductor, wherein the flexible circuit board is arranged between the inner liner outer surface and the insulation inner surface, wherein the flexible circuit board is wound at least in sections in a spiral manner in turns around the inner liner and wherein the inner liner and the insulation are connected to one another, in particular integrally, in the spaces between the turns of the flexible circuit board;
b) creating at least one contact opening in the vicinity of the distal catheter body end, wherein, to create the contact opening, parts of the insulation layer and of any insulating layers of the flexible circuit board are removed so that the at least one electrical conductor can be selectively electrically contacted via the contact opening;
c) providing a contact means;
d) applying the contact means into the at least one contact opening;
e) providing at least one electrode;
f) positioning the electrode at the position of the contact opening in contact with the contact means in the contact opening;
g) targeted heating of the electrode and of the contact means in order to secure the electrode to the electrical conductor via the contact means in an electrically conductive manner.

In a preferred embodiment of the method, the targeted heating in method step g) raises the electrode and the contact means to a fixing temperature which is higher than a temperature of the regions of the catheter body surrounding the contact opening. This embodiment is a second embodiment of the invention, which is preferably dependent upon the first embodiment of the invention.

In a preferred embodiment of the method, the targeted heating in method step g) is carried out without convection. This embodiment is a third embodiment of the invention, which is preferably dependent upon the first or second embodiment of the invention.

In a preferred embodiment of the method, the electrode consists of a metal. This embodiment is a fourth embodiment of the invention, which is preferably dependent upon one of the preceding embodiments of the invention.

In a preferred embodiment of the method, the metal is selected from the group consisting of platinum, iridium, tantalum, palladium, titanium, iron, gold, molybdenum, niobium, tungsten, nickel, chromium, cobalt, steel, nitinol and alloys comprising at least one of these metals. This embodiment is a fifth embodiment of the invention, which is preferably dependent upon the fourth embodiment of the invention.

In a preferred embodiment of the method, the targeted heating in method step g) is achieved by means of targeted irradiation of the electrode with electromagnetic radiation. This embodiment is a sixth embodiment of the invention, which is preferably dependent upon one of the preceding embodiments of the invention.

In a preferred embodiment of the method, electromagnetic radiation is generated by means of a laser. This embodiment is a seventh embodiment of the invention, which is preferably dependent upon the sixth embodiment of the invention.

In a preferred embodiment of the method, the laser is an IR laser. This embodiment is an eighth embodiment of the invention, which is preferably dependent upon the seventh embodiment of the invention.

In a preferred embodiment of the method, the targeted heating in method step g) is carried out by means of induction. This embodiment is a ninth embodiment of the invention, which is preferably dependent upon one of the preceding embodiments of the invention.

In a preferred embodiment of the method, the targeted heating in method step g) is carried out via direct contact of the electrode with a heating element. This embodiment is a tenth embodiment of the invention, which is preferably dependent upon one of the preceding embodiments of the invention.

In a preferred embodiment of the method, the heating element is a soldering iron. This embodiment is an eleventh embodiment of the invention, which is preferably dependent upon the tenth embodiment of the invention.

In a preferred embodiment of the method, the contact means is a solder, a sintering paste or a conductive paste. This embodiment is a twelfth embodiment of the invention, which is preferably dependent upon one of the preceding embodiments of the invention.

In a preferred embodiment of the method, the targeted heating in method step g) is carried out to a temperature in a range between 200° C. and 280° C. This embodiment is a thirteenth embodiment of the invention, which is preferably dependent upon one of the preceding embodiments of the invention.

In a preferred embodiment of the method, the targeted heating in method step g) is carried out to a temperature which is below the melting temperature of the electrode. This embodiment is a fourteenth embodiment of the invention, which is preferably dependent upon one of the preceding embodiments of the invention.

With respect to the embodiments described herein, the elements of which “have,” or “comprise,” a particular feature (for example, a material), in principle, a further embodiment is always contemplated in which the relevant element consists solely of the feature, i.e., does not comprise any other constituents. The word “comprise” or “comprising” is used herein synonymously with the word “have” or “having.”

In one embodiment, if an element is denoted by the singular, an embodiment is also contemplated in which more than one such element is present. The use of a term for an element in the plural in principle also encompasses an embodiment in which only a single corresponding element is included.

Unless otherwise indicated or clearly excluded from the context, it is possible in principle, and is hereby clearly contemplated, that features of different embodiments may also be present in the other embodiments described herein. Likewise, it is contemplated in principle that all features described herein in connection with a method are also applicable to the products and devices described herein, and vice versa.

All such combinations considered are not explicitly listed in all instances, simply in order to keep the description brief. Technical solutions known to be equivalent to the features described herein are also intended in principle to be encompassed by the scope of the invention.

In the present description, specifications of ranges also contain the values specified as limits. A specification of the type “in the range from X to Y” with respect to a quantity A consequently means that A can take the values X, Y and values between X and Y. Ranges which are limited on one side of the type “up to Y” for a size A, accordingly, mean a value Y and less than Y.

Some of the features described are associated with the term “substantially”. The term “substantially” is to be understood in such a way that, under real conditions and manufacturing techniques, a mathematically exact interpretation of terms such as “superimposition”, “perpendicular”, “diameter” or “parallelism” can never be given exactly, but only within certain manufacturing error tolerances. For example, “substantially perpendicular axes” enclose an angle of 85 degrees to 95 degrees relative to one another, and “substantially equal volumes” comprise a variation of up to 5% by volume. For example, a “device consisting substantially of plastics” comprises a plastics content of ≥95 to ≤100% by weight. For example, a “substantially complete filling of a volume B” comprises a filling of ≥95 to ≤100% by volume of the total volume of B.

### SUMMARY

A first subject of the invention relates to a method for producing a catheter comprising the following method steps:
a) providing a catheter body comprising a hollow cylindrical inner liner having an inner liner outer surface, the inner liner extending in a longitudinal direction from a proximal catheter body end to a distal catheter body end of the catheter body,
   an outer insulation having an inner insulation surface, the insulation coaxially surrounding the inner liner,
   a flexible circuit board which comprises at least one electrical conductor, wherein the flexible circuit board is arranged between the inner liner outer surface and the insulation inner surface, wherein the flexible circuit board is wound at least in sections in a spiral manner in turns around the inner liner and wherein the inner liner and the insulation are connected to one another, in particular integrally, in the spaces between the turns of the flexible circuit board;
b) creating at least one contact opening in the vicinity of the distal catheter body end, wherein, to create the contact opening, parts of the insulation layer and of any insulating layers of the flexible circuit board are removed so that the at least one electrical conductor can be selectively electrically contacted via the contact opening;
c) providing a contact means;
d) applying the contact means into the at least one contact opening;
e) providing at least one electrode;
f) positioning the electrode at the position of the contact opening in contact with the contact means in the contact opening;
g) targeted heating of the electrode and of the contact means in order to secure the electrode to the electrical conductor via the contact means in an electrically conductive manner.

The method serves to produce a catheter using a catheter body. A catheter body is the elongate, tube-like portion of the catheter which, depending on the application of the catheter, is equipped with an electrode or a plurality of electrodes, at least at a distal catheter body end, preferably both at the distal and at a proximal catheter body end axially opposite the distal catheter body end. The electrodes at the proximal end of the catheter body are sometimes called connectors.

In a method step a), a catheter body is provided.

The catheter body comprises a hollow cylindrical inner liner with a radially outer inner liner outer surface. The inner liner extends axially from a proximal catheter body end to a distal catheter body end and radially surrounds at least one lumen extending axially through the catheter. The lumen is used to pass and/or introduce various devices or fluids. For example, the lumen is used to insert a guide wire, which facilitates the insertion of the catheter into the patient.

The inner liner comprises an inner liner inner diameter which is, for example, in a range of 0.75 to 4 mm, and an inner liner outer diameter which is, for example, in a range of 1 to 4.5 mm. Depending on the application, the inner liner can comprise different lengths. For example, the inner liner comprises a length in a range from 30 cm to 200 cm.

The inner liner can be made of a variety of different materials, wherein the use of a flexible material or a flexible material combination is preferred. For example, the inner liner is made of a polymer that is selected from the group of silicones, polyolefins (e.g. polyethylene), polyether block amide (e.g. PEBAX©), polyurethanes, polyimides, polyamides, polyaryletherketones (e.g. polyetheretherketone), fluorinated polymers (e.g. selected from the group of ethylene tetrafluoroethylene, polytetrafluoroethylene, perfluoroalkoxyalkanes, polyvinylidene fluorides, fluorinated ethylene propylene and mixtures thereof) and mixtures thereof. According to a preferred embodiment, the inner liner comprises a polymer that is selected from the group of silicones, polyolefins (e.g. polyethylene), polyether block amide (e.g. PEBAX©), polyurethanes, polyimides, polyamides, polyaryletherketone (e.g. polyetheretherketone), fluorinated polymers (e.g. selected from the group of ethylene tetrafluoroethylene, polytetrafluoroethylene, perfluoroalkoxyalkanes, polyvinylidene fluorides, fluorinated ethylene propylene and mixtures thereof) and mixtures thereof.

An outer insulation having an inner insulation surface is arranged coaxially around the inner liner, the inner insulation surface facing the outer liner surface. The outer insulation is preferably the radially outer layer of the catheter and serves in particular to electrically insulate the catheter from the outside and to mechanically protect the catheter. Preferably, the outer insulation has an outer diameter in a range of 200 μm to 5000 μm, more preferably 300 μm to 3000 μm and most preferably 500 μm to 1500 μm. In an exemplary embodiment, the outer diameter of the outer insulation is about 700 μm. It is understood that the outer diameter of the outer insulation can also correspond to the outer diameter of the catheter. The outer insulation preferably has a wall thickness in the range of 2 μm to 300 μm, particularly preferably from 5 μm to 150 μm, and most preferably from 20 μm to 100 μm. According to a preferred embodiment of the present invention, the outer insulation has an outer diameter in a range from 300 μm to 3000 μm, particularly preferably from 500 μm to 1500 μm, and a wall thickness in a range from 5 μm to 150 μm, particularly preferably from 20 μm to 100 μm. In an exemplary embodiment, the wall thickness is approximately 60 μm. The outer insulation preferably consists of a polymer that is selected from the group of silicones, polyolefins (e.g. polyethylene), polyether block amide (e.g. PEBAX©), polyurethanes, polyimides, polyamides, polyaryletherketones (e.g. polyetheretherketone), fluorinated polymers (e.g. selected from the group of ethylene tetrafluoroethylene, polytetrafluoroethylene, perfluoroalkoxyalkanes, polyvinylidene fluorides, fluorinated ethylene propylene and mixtures thereof) and mixtures thereof. According to a preferred embodiment, the outer insulation comprises a polymer that is selected from the group of silicones, polyolefins (e.g., polyethylene), polyurethanes, polyimides, polyamides, polyaryletherketone (e.g., polyetheretherketone), fluorinated polymers (e.g., selected from the group of ethylene tetrafluoroethylene, polytetrafluoroethylene, perfluoroalkoxyalkanes, polyvinylidene fluorides, fluorinated ethylene propylene, and mixtures thereof), and mixtures thereof, and has an outer diameter in the range of 300 to 3000 μm.

A flexible circuit board is arranged radially between the inner liner and the outer insulation, i.e. between the inner insulation surface and the outer inner liner surface. A flexible circuit board, sometimes also called flex circuit ribbon, is a circuit board comprising or consisting of flexible, bendable materials that allow the circuit board to be bent or folded or wrapped into different shapes. Typically, the structure of a typical flexible circuit board comprises several layers. The core element is one or more electrically conductive layers, for example made of a metal foil or a printed metal layer which can contain a specific pattern, preferably comprise copper or consisting of copper, depending on the type of application. This layer or a conductor track within this layer acts as the electrical conductor of the flexible circuit board. The electrically conductive layers are usually embedded between electrically insulating layers made of flexible insulation material such as polyimides which can also provide mechanical protection. Flexible circuit boards offer advantages when used in catheters, in particular due to their small space requirement, low weight and simplified installation. Installing individual conductive wires, in particular through the lumen of the inner liner, is unnecessary due to the use of a flexible circuit board.

The flexible circuit board of the catheter body according to the invention comprises at least one electrical conductor. For example, the flexible circuit board comprises two to 20 electrical conductors, each of which can be electrically connected to one or more electrodes. For example, the flexible circuit board comprises 12 electrical conductors which can be selectively connected to 12 electrodes of the catheter. Preferably, the electrical conductors are electrically isolated from each other. More preferably, the flexible circuit board comprises at least two insulating layers which electrically insulate the at least one electrical conductor from the outside at least over long sections of the flexible circuit board. The flexible circuit board can have contact surfaces at certain positions which make the electrical conductor electrically contactable from the outside without the need to remove part of the insulating layer beforehand.

The flexible circuit board is arranged at least in sections, preferably substantially completely over the entire length of the flexible circuit board, in a spiral manner in turns around the inner liner. The flexible circuit board therefore extends axially in spiral turns between the inner liner and the outer insulation of the catheter body, preferably along substantially the entire length of the catheter body.

In order to provide a catheter body that is as mechanically stable as possible, the inner liner and the outer insulation are integrally bonded to one another in the spaces between the turns of the flexible circuit board, for example by fusing or using a bonding material such as an adhesive. This has the advantage, for example, that the flexible circuit board is at least partially spatially secured by this integral bond and cannot, for example, slip between the inner liner and the outer insulation, which could be the case with layers not connected to each other. To facilitate the production of the catheter body, the flexible circuit board can be wrapped around the inner liner as desired and secured to the inner liner outer surface using an adhesive such as an adhesive or an adhesive strip, for example a double-sided adhesive tape, before the outer insulation in the form of a hollow cylindrical hose or tube is pulled on, or in other words, pulled over, the inner liner and the flexible circuit board. A temperature treatment afterwards can form the integral bond between the inner liner and the outer insulation in the spaces between the turns of the flexible circuit board. Preferably, the flexible circuit board itself does not comprise a layer which, due to this temperature treatment itself, forms an integral bond with the inner liner or the outer insulation. For example, the flexible circuit board comprises, at least on the outside, only polymer layers made of a polyimide.

In a method step b), at least one contact opening is created in the vicinity of the distal catheter body end. “In the vicinity of the distal catheter body end” means that a position lies within the last 30%, preferably the last 20%, most preferably the last 10% of the end of the catheter body, and “in the proximity of the proximal catheter body end” means that a position lies within the first 30%, preferably the first 20%, most preferably the first 10% of the end of the catheter body.

To create the contact opening, part of the outer insulation and, if necessary, part of the insulating layer of the flexible circuit board is removed at the corresponding position so that the at least one electrical conductor is exposed radially from the outside via the contact opening to be electrically contactable. The removal of parts of the insulating layer of the flexible circuit board is in particular necessary if the flexible circuit board does not already comprise an accessible electrical conductor at the position of the contact opening or has a contact surface via which the electrical conductor can be electrically contacted from the outside without removing parts of the insulating layer. At the position of the contact opening, an electrode can be selectively, or in other words specifically, electrically contacted with the electrical conductor over the course of the subsequent method. If the catheter body is to comprise more than one electrode, in particular at the distal catheter body end, it is preferred that a separate contact opening is created for each of these electrodes, wherein one of the electrical conductors of the flexible circuit board is selectively electrically contacted via each of these contact openings. For this purpose, the flexible circuit board preferably comprises at least as many electrical conductors as electrodes are to be attached to the distal catheter body end.

The contact opening can be created in different ways. For example, the contact opening can be created by cutting, punching or, preferably, by laser ablation.

In a method step c), a contact means is provided. The contact means serves to electrically contact the electrical conductor of the flexible circuit board with an electrode via the contact opening. The contact means is a conductive material, at the latest after a temperature treatment, which can simultaneously, at least after a temperature treatment, mechanically secure the electrical conductor to the electrode. The contact means also serves to bridge the depth of the contact opening, which corresponds at least to the thickness of the outer insulation, so that neither the electrical conductor nor the electrode to be connected thereto have to overcome the radial depth of the contact opening alone. Without contact means, for example, the electrical conductor would have to be at least partially pulled radially outwards, and/or the electrode would have to have a radially inwardly directed projection which can overcome at least significant parts of the depth of the contact opening.

The contact means can be designed in different ways. The contact means is preferably a solder, for example in the form of a solder paste, a conductive paste or a sintering paste. This type of contact means allowing an electrical connection between the electrical conductor and the electrode and, after a temperature treatment, can simultaneously serve as a mechanical fastening agent between the electrical conductor and the electrode.

In a method step d), the contact means is applied into the at least one contact opening. The type of application depends on the type of contact means. For example, pasty contact means can be applied by means of a printing method or by injection into the contact opening. The volume of contact means applied into the contact opening can depend on the type of contact means. For example, the contact means can substantially completely fill the contact opening. Completely filling the contact opening with contact means can ensure that a well-formed electrical connection between the electrical conductor and the electrode can be achieved in the further course of the method, which at the same time simultaneously allows a stable mechanical fixation of the electrode to the electrical conductor.

In a method step e), at least one electrode is provided. The exact number of electrodes depends on the use of the catheter. Preferably, a maximum of as many electrodes as electrical conductors are provided so that each of the electrodes can be electrically connected to an electrical conductor during the method.

Preferably, the electrodes are ring electrodes. Further preferably, all provided electrodes are ring electrodes.

The electrode can comprise a plurality of different materials or consist of different materials. Preferably, the electrode comprises a metal that is selected from the group consisting of platinum, iridium, tantalum, palladium, titanium, iron, gold, molybdenum, niobium, tungsten, nickel, chromium, cobalt, steel, nitinol, alloys of any of these metals, and composites of any of these metals. Stainless steel is suitable as an electrode, for example stainless steel AISI 316L, stainless steel AISI 301 or stainless steel AISI 304. Platinum and platinum alloys such as Pt/Ir 10 or Pt/Ir 20 or nickel-cobalt alloys such as MP35N are also suitable as electrodes.

The choice of metal for the electrodes (and for the electrical conductors) can depend on the use of the catheter according to the invention. However, it should be understood that the application of the catheter is not limited by the use of a particular metal.

The electrode can also comprise a coating. Suitable coatings are metal nitrides such as TiN, metal oxides such as IrO₂ or conductive polymers. The surface of the electrode can also be surface-structured, e.g. laser-structured.

The electrode, preferably a ring electrode, can have an outer diameter in the range of 200 to 5000 μm, in one embodiment in the range of 300 to 3000 μm and in one embodiment in the range of 500 to 1500 μm. The electrode can have a wall thickness in the range of 10 to 200 μm, in one embodiment 10 to 100 μm, and in one embodiment 30 to 70 μm. Furthermore, the electrode can have a length in the range of 200 to 5000 μm, in one embodiment 300 to 3000 μm, and in one embodiment in the range of 500 to 1500 μm. According to one embodiment, the electrode has an outer diameter in the range of 300 to 3000 μm and in one embodiment in the range of 500 to 1500 μm, a wall thickness in the range of 10 to 100 μm and in one embodiment of 30 to 70 μm, and a length in the range of 300 to 3000 μm and in one embodiment in the range of 500 to 1500 μm.

In a method step f), the electrode is positioned at the position of the contact opening in contact with the contact means in the contact opening. For example, the electrode is a ring electrode, and positioning involves axially pulling the ring electrode onto the catheter body to the position of the desired contact opening. If in this example the contact opening is filled with sufficient contact means, pulling the electrode to the appropriate position can be sufficient to establish contact between the electrode and the contact means. In further embodiments, it can be necessary to displace the electrode radially inward toward the contact means. In the case of a ring electrode as the electrode, it can be necessary, for example, to compress the ring electrode radially in order to establish a corresponding contact between the electrode and the contact means. The contact between the electrode and the contact means serves to electrically conductively secure the electrode to the electrical conductor over the further course of the method.

In a method step g), a targeted, or in other words a selective, heating of the electrode and the contact means takes place. Other regions of the catheter body are not specifically heated and therefore remain at a lower temperature than the specifically heated electrode and the contact means. It goes without saying that the heated electrode and/or the heated contact means always also releases a certain amount of heat to the adjacent regions of the catheter body, but the targeted heating is preferably carried out in such a way that the heat input or heat development is concentrated substantially on the electrode and/or the contact means. For example, the material at an axial distance of 5 mm from the electrode heats up by no more than 100° C. relative to the previous temperature of this region, for example, by no more than 100° C. above room temperature.

Through the targeted heating of the electrode and the contact means, the remaining part of the catheter body, possibly with the exception of further sections with additional electrodes, remains at the previous temperature, preferably in a temperature range between 18° C. and 30° C. Accordingly, the majority of the catheter body, in particular temperature-sensitive parts such as the inner liner, the outer insulation, and the flexible circuit board, which can all comprise polymer, are protected from high temperature input. This significantly reduces the risk of blistering and/or other deformations of the utilized polymers, which can limit the functionality of the final catheter.

Through targeted heating, the electrode is secured to the electrical conductor in an electrically conductive manner. This means that the contact means forms a, preferably securely adhering, connection between the electrode and the electrical conductor, which is electrically conductive. Depending on the type of contact means, it can only develop its full conductivity through targeted heating, for example in the case of contact means in the form of pastes such as solder pastes or conductive pastes, or the contact means is already in possession of its full, specific conductivity before targeted heating, for example in the case of contact means in the form of pure solder alloys, i.e. without the addition of, for example, organic solvents which would first have to evaporate during targeted heating in order to achieve full conductivity.

The contact means can be heated indirectly by heating the electrode, or the electrode and contact means can both be heated directly by targeted heating. In indirect heating, the contact means is heated by direct physical contact of the contact means with the heating electrode. In direct heating, the contact means is heated at least partially without using direct physical contact with the electrode. In direct heating, the type of targeted heating is chosen in such a way that the contact means itself is heated in a targeted manner.

One embodiment of the method is characterized in that the targeted heating in method step g) raises the electrode and the contact means to a fixing temperature which is higher than the temperature of the regions of the catheter body surrounding the contact opening.

For example, the temperature of the surrounding regions of the catheter body that are located at an axial distance of 5 mm around the targeted heated electrode remains in a range between 18° C. and 130° C., preferably between 18° C. and 100° C., more preferably between 18° C. and 80° C. It is understood that the temperature of regions that are even further away from a targeted heated electrode is lower than that of regions that are closer to the corresponding electrode.

Targeted heating can be effected in different ways.

One embodiment of the method is characterized in that the targeted heating in method step g) is carried out without convection. In this context, without convection means that the targeted heating does not occur via heat transport via fluids, i.e. gases or liquids. For example, targeted heating is not carried out in an oven into which the catheter body, including electrodes and contact means, is inserted. Heating by convection is not sufficiently targeted, at least without considerable technical effort, so that the regions surrounding the electrode would be exposed to excessively high temperature load, or even to the same temperature load as the contact means. This can lead to undesirable deformation.

One embodiment of the method is characterized in that the electrode is made of metal. This is preferred due to the good thermal conductivity of metals since targeted heating of the electrode via direct physical contact with the contact means can also heat it. With metallic electrodes, it is therefore not necessary to heat the contact means itself; it can be sufficient to simply heat the electrode in a targeted manner, which then transfers the heat to the contact means.

The electrode can be made of different metals.

One embodiment of the method is characterized in that the metal is selected from the group consisting of platinum, iridium, tantalum, palladium, titanium, iron, gold, molybdenum, niobium, tungsten, nickel, chromium, cobalt, steel, nitinol and alloys comprising at least one of these metals.

The electrode can be coated and/or structured to adapt its electrical properties.

An embodiment of the method is characterized in that the electrodes comprise a coating with an electrically conductive polymer. This coating can be applied at least to the inner side facing the inner liner, to the radially outer side, or to both sides.

The coating may, for example, comprise or consist of a poly-3,4-ethylenedioxythiophene (PEDOT). Other possible coatings include iridium oxides or titanium nitrides, for example.

The targeted heating in method step g) can be carried out in different ways, wherein both a single targeted heating process and a combination of several targeted heating processes can be used. Preferably, only one single process of targeted heating is used.

One embodiment of the method is characterized in that the targeted heating in method step g) is carried out by means of targeted irradiation of the electrode with electromagnetic radiation. In this embodiment, the electrode is specifically irradiated with electromagnetic radiation, while the regions of the catheter body surrounding the electrode, in particular the outer insulation, are not irradiated with electromagnetic radiation. The electromagnetic radiation is at least partially absorbed by the electrode, preferably in the form of a metallic electrode, whereby it is heated in a targeted manner, preferably to the fixing temperature. The electrode which heats up in this way also heats the contact means through direct physical contact, which subsequently secures the electrode to the electrical conductor in an electrically conductive manner.

The use of electromagnetic radiation can be particularly easily directed at the electrode, for example by means of apertures or mirrors. Furthermore, it is possible to heat a plurality of electrodes simultaneously in a targeted manner, which can increase the productivity of the method.

The utilized wavelength of electromagnetic radiation must be selected depending on the material of the electrode.

One embodiment of the method is characterized in that the electromagnetic radiation is generated by means of a laser. Lasers are in particular also suitable for the targeted heating of smaller electrodes and generally for heating them particularly quickly and efficiently.

One embodiment of the method is characterized in that the laser is an IR laser.

One embodiment of the method is characterized in that the targeted heating in method step g) is carried out by means of induction, in particular electromagnetic induction. For this purpose, an alternating magnetic field is generated in the region of the electrode and/or the contact means by means of an inductor, preferably a type of coil, which induces a current in the electrode and/or the contact means and, due to the electrical resistance of the material of the electrode and/or the contact means, generates the targeted heat for the electrically conductive securing of the electrode to the electrical conductor. Depending on the type of contact means, it can be heated directly to the required temperature, especially the fixing temperature, by means of induction. Preferably, the electrode is a metallic electrode so that at least this is also heated in a targeted manner by means of induction, and the contact means is heated at least partially indirectly via direct physical contact with the heating electrode.

The alternating magnetic field used to exploit the inductive effect is only generated in a targeted manner in the region of the electrode or the contact means so that the regions of the catheter body surrounding the contact opening do not heat up substantially.

An embodiment of the method is characterized in that the targeted heating in method step g) is carried out via direct contact of the electrode with a heating element. For this purpose, the heating element is brought into direct physical contact with the electrode, preferably in the form of a metallic electrode. Regarding timing, the heating element itself can only be heated after direct contact with the electrode, but due to the efficiency of the method, it is preferred if the heating element has already been brought to its operating temperature before direct contact with the electrode.

The heated heating element heats the electrode in a targeted manner by means of conduction, which heats the contact means in a targeted manner through direct physical contact therewith.

In one embodiment of the method, a plurality of electrodes with a plurality of heating elements are each heated in a targeted manner, which can increase the efficiency of the method.

Depending on the scale of the electrode and the utilized materials, in particular the contact means utilized, the design of the heating element can be different. Typically, a heating element converts electrical energy into heat which can be transferred to the electrode and then to the contact means through direct contact therewith.

One embodiment of the method is characterized in that the heating element is a soldering iron.

Depending on the application of the catheter, the contact means can be designed differently.

One embodiment of the method is characterized in that the contact means is a solder, particularly a solder alloy, a sintering paste, a conductive paste or a combination of the aforementioned.

A solder is preferably a metallic alloy with a melting temperature which is lower than the melting temperature of the electrode and the electrical conductor. After achieved heating and subsequent cooling, the solder secures the electrode and the electrical conductor in an electrically conductive manner, preferably by at least partially filling the contact opening between the electrode and the electrical conductor. Before the method step, the solder can be present as a pure alloy or as a solder paste. A solder paste comprises the solder alloy in particle form dispersed in an organic medium. Through the targeted heating in method step g), the organic medium preferably evaporates substantially completely.

The solder is preferably selected from the group consisting of tin, silver, copper, and/or indium-containing metal alloys. For example, the solder comprises a SAC305 alloy.

A sintering paste comprises metal particles in an organic medium. During the targeted heating in method step g), the metal particles sinter at least partially together so that an electrical connection is formed between the electrode and the electrical conductor. Through the targeted heating in method step g), the organic medium preferably evaporates substantially completely.

The metal particles of the sintering paste are preferably selected from the group consisting of silver, platinum and gold.

A conductive paste preferably comprises electrically conductive particles, preferably metal particles or conductive carbon-containing compounds such as carbon nanotubes or graphene, dispersed in an organic medium. Through the targeted heating in method step g), at least parts of the organic medium harden and electrically contact the electrode with the electrical conductor. The dispersed particles ensure an electrically conductive connection between the electrode and the electrical conductor.

The metal particles of the conductive paste are preferably selected from the group consisting of silver, platinum and gold.

The organic medium remains at least partially in the contact opening after method step g). For example, the organic medium is a polymer in which the conductive particles are dispersed. In a further embodiment, the organic medium of the conductive paste contains a polymer dissolved in an organic solvent, wherein the curing of the conductive paste takes place by at least partial, preferably substantially complete, evaporation of the solvent so that the previously dissolved polymer cures in the contact opening. In a further embodiment, the organic medium of the conductive paste contains monomers which, through targeted heating, polymerize into a polymer which secures the electrode to the electrical conductor.

The polymer of the organic medium is preferably selected from the group consisting of silicones, polyolefins (e.g., polyethylene), polyether block amides (e.g., PEBAX©), polyurethanes, polyimides, polyamides, polyarylether ketones (e.g., polyether ether ketone), fluorinated polymers (e.g. selected from the group of ethylene tetrafluoroethylene, polytetrafluoroethylene, perfluoroalkoxyalkanes, polyvinylidene fluorides, fluorinated ethylene propylene and mixtures thereof) and mixtures thereof.

Depending on the embodiment of the catheter and in particular of the contact means, the targeted heating in method step g) can be carried out to different temperatures.

An embodiment of the method is characterized in that the targeted heating in method step g) is carried out to a temperature, in particular a fixing temperature, in a range between 200° C. and 280° C.

An embodiment of the method is characterized in that the targeted heating in method step g) is carried out to a temperature which lies below the melting temperature of the electrode, and preferably also below the melting temperature of the electrical conductor. This ensures that the electrode does not become deformed, such as a bulge, which could impair the functionality of the catheter. For example, an elevation of the electrode can increase the patient's risk of injury when using the catheter.

Preferably, the targeted heating is carried out to a temperature which is at least 500° C., preferably at least 700° C., more preferably at least 900° C., below the melting temperature of the electrode, and preferably also that of the electrical conductor.

The method steps of the method are preferably carried out in the indicated order, but can, if technically possible, be carried out in a different order.

If the catheter comprises a plurality of electrodes, the method can be designed such that the targeted heating in method step g) is carried out simultaneously with all these electrodes. In further embodiments, the electrodes can also be heated sequentially in a targeted manner, wherein the previous method steps, in particular method steps b) to f), can also be carried out simultaneously or sequentially.

### BRIEF DESCRIPTION OF THE DRAWINGS

The invention is further illustrated by way of example below by means of figures. The invention is not limited to the figures in which:

FIG. 1 is an example of a flow chart of a method for producing a catheter;

FIG. 2 is an example of schematic components for use in a method for producing a catheter, in a perspective side view; and,

FIGS. 3a-f are exemplary method steps or intermediate products 3a to 3f of the method from FIG. 1 shown with the components from FIG. 2 in schematic longitudinal sections.

### DETAILED DESCRIPTION OF THE INVENTION

FIG. 1 shows a flow chart of an exemplary method 200 for producing a catheter comprising the method steps 210 to 270.

In a method step 210, a catheter body is provided. The catheter body comprises a hollow cylindrical inner liner with a radially outer inner liner outer surface, an outer insulation with a radially inner insulation inner surface, wherein the outer insulation coaxially surrounds the inner liner.

A flexible circuit board is arranged in spiral turns between the inner liner outer surface and the insulation inner surface. The flexible circuit board comprises at least one electrical conductor. In the spaces between the turns of the flexible circuit board, the inner liner is integrally bonded to the outer insulation.

For example, the catheter body can be provided in the following way:

After the inner liner has been provided, the provided flexible circuit board is spirally wound onto the inner liner outer surface. It can be advantageous for the flexible circuit board to be attached to the inner liner outer surface so that there is no accidental slipping of the flexible circuit board on the inner liner. For example, this attachment can be performed by means of double-sided adhesive tape.

This is followed by a coaxial sheathing of the inner liner and the flexible circuit board wound on the inner liner. For example, for this purpose, a hollow cylindrical outer insulation can be pushed onto the inner liner. In order to form the integral bond between the inner liner and the outer insulation, the structure can be heated. The integral bond between the turns of the flexible circuit board can at least give the catheter body stability against displacement of the flexible circuit board between the inner liner and the outer insulation.

In a method step 220, at least one contact opening is created in the vicinity of a distal catheter body end. The contact opening serves to electrically expose the at least one electrical conductor of the flexible circuit board so that it can be electrically contacted from outside the catheter body. To do this, at least parts of the electrical conductor above the position to be contacted are removed. If the electrical conductor is covered by one or more insulating layers of the flexible circuit board at this position, the creation of the contact opening also includes the removal of this/these insulating layer(s). The contact opening is preferably created by laser ablation. Preferably, the electrical conductor comprises a contact surface at the corresponding position, which can be freely electrically contacted from outside the flexible circuit board so that removal of insulating layers of the flexible circuit board is not necessary.

In a method step 230, a contact means is provided. The contact means serves as an electrically conductive link between the electrical conductor and an electrode to be electrically contacted therewith. The contact means is preferably a solder, a sintering paste or a conductive paste.

In a method step 240, the contact means is applied into the at least one contact opening. Preferably, the contact means is flowable, for example pasty or liquid, or is provided in a flowable manner, for example by heating, such as heating a solder to or above its melting temperature. This has the advantage that the contact means can be easily applied into the contact opening, for example by pouring or dispensing.

In a method step 250, at least one electrode is provided. Preferably, the electrode comprises a metal. More preferably, the electrode is a ring electrode, preferably a metallic ring electrode.

In a method step 260, the electrode is positioned at the position of the contact opening in contact with the contact means in the contact opening. In a preferred embodiment of the method, the electrode is a ring electrode, and positioning the same comprises compressing the ring electrode to a smaller diameter (often referred to as crimping).

In a method step 270, targeted heating of the electrode and also direct or, at least partially, indirect heating of the contact means in the contact opening is carried out. The targeted heating serves to secure the electrode to the electrical conductor via the contact means in an electrically conductive manner. For example, targeted heating serves to melt, sinter, polymerize and/or evaporate volatile substances, such as organic solvents, of the contact means.

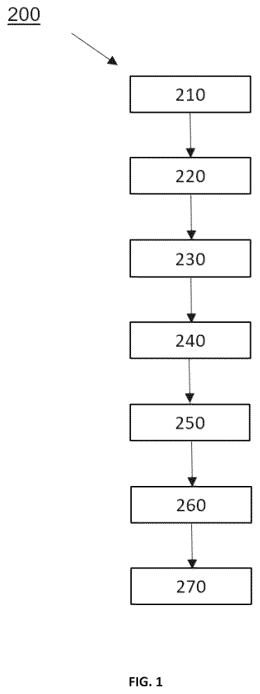

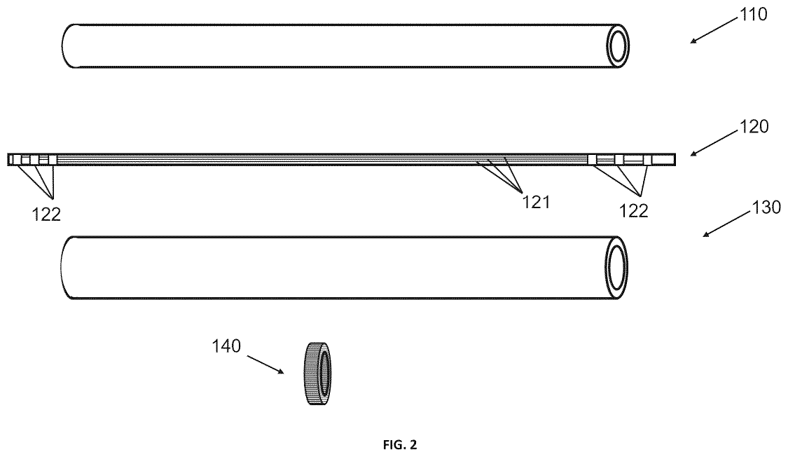

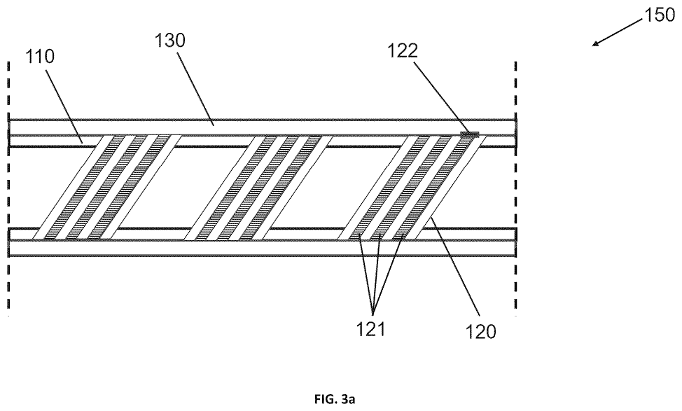

FIG. 2 shows various exemplary components of the method 200 for producing a catheter in a perspective side view. Shown are a hollow cylindrical inner liner 110, a hollow cylindrical outer insulation 130, an electrode 140 in the form of a metallic ring electrode, as well as a flexible circuit board 120. The flexible circuit board 120 comprises three electrical conductors 121, each extending from one axial end of the flexible circuit board 120 to an opposite axial end of the flexible circuit board 120. At both ends of the flexible circuit board 120, the electrical conductors 121 selectively terminate in each case in three contact surfaces 122 which are designed to selectively electrically contact one of the electrical conductors 121 at the distal end and/or at the proximal end of the flexible circuit board 120. In other words, each of the electrical conductors 121 extends from one of the contact surfaces 122 at one end of the flexible conductor 120 to one of the contact surfaces 122 at the opposite end of the flexible conductor 120. In the shown generally preferred embodiment of the flexible circuit board 120, the electrical conductors 121, including the contact surfaces 122 at both ends thereof, are electrically insulated from the outside, preferably by embedding in one or more electrically insulating layers. The contact surfaces 122, on the other hand, can be electrically contacted from the outside and are therefore freely accessible. The inner and outer diameters of the inner liner 110, outer insulation 130 and electrode 140 are matched to one another in such a way that the outer insulation 130 can be pushed coaxially onto the inner liner 110, and the electrode 140 can be pushed coaxially onto the outer insulation 130.

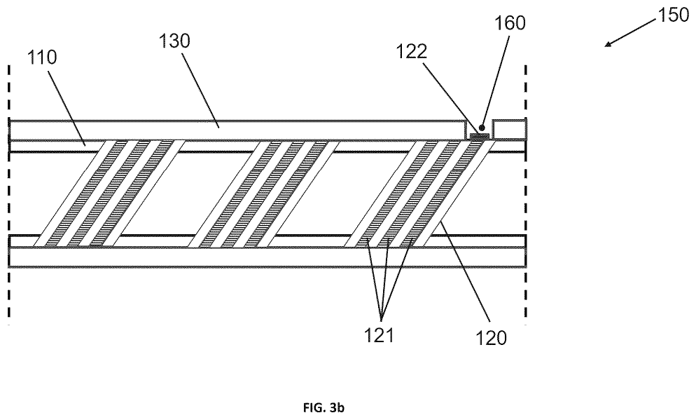

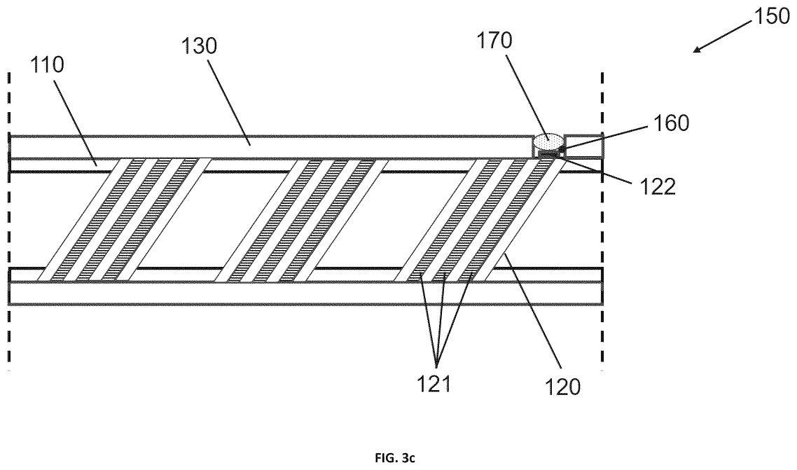

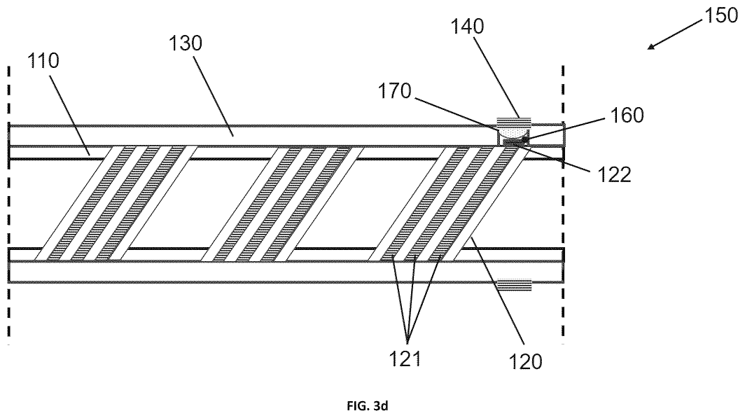

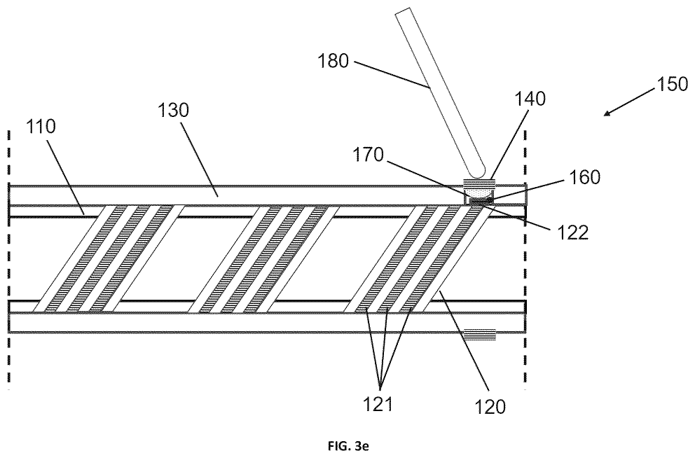

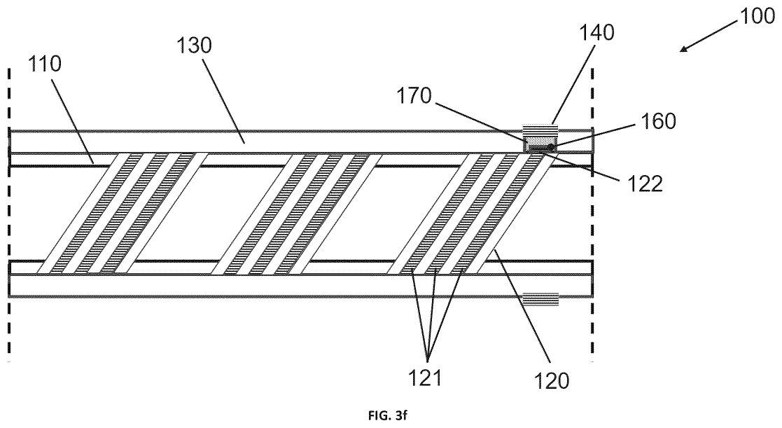

FIGS. 3a-f show various intermediate products or method steps of the method from FIG. 2 based on the components of FIG. 2.

FIG. 3a shows a section of a provided catheter body 150 in a schematic longitudinal section. The catheter body 150 includes the inner liner 110, the outer insulation 130 and the flexible circuit board 120, wherein this is spirally wound in multiple turns around the inner liner 110. For better clarity, the flexible circuit board 120 is shown in a top view. One of the electrical conductors 121 has one of its contact surfaces 122 in the shown section. In the regions between the turns of the flexible circuit board 120, the inner liner 110 and the outer insulation 130 are integrally connected to one another (not explicitly shown).

FIG. 3b shows the catheter body 150 in FIG. 3a, wherein a contact opening 160 has been created at the position of the contact surface 122 of one electrical conductor 120 by removing parts of the outer insulation 130. The corresponding electrical conductor 120 can be electrically contacted from the outside via the contact opening 160, in particular via its contact surface 122 present at the position of the contact opening 160.

FIG. 3c shows the catheter body 150 from 3b, wherein a contact means 170 in the form of a solder has been introduced into the contact opening 160. In the embodiment shown, a molten solder has been applied into the contact opening 160. In further embodiments not shown, the contact means 170 is, for example, a conductive paste which is injected into the contact opening 160.

FIG. 3d shows the catheter body 150 of FIG. 3c, wherein the provided electrode 140 in the form of a ring electrode from FIG. 2 has been positioned at the position of the contact opening 160 in contact with the contact means 170. For this purpose, the electrode 140 was pushed axially from one end of the catheter body 150 onto the same until it reached the corresponding position and then compressed radially. The radial compression has brought the electrode 140 into contact with the contact means 170.

FIG. 3e shows the catheter body of FIG. 3d during targeted heating of the electrode 140 and the contact means 170 in the contact opening 160. In the shown embodiment of the method 200, the targeted heating is carried out by means of a heating element 180 in the form of a soldering iron. For this purpose, the heating element 140 has been brought into direct, in particular direct physical, contact with the electrode 140. The heating electrode 140 in the form of a metallic ring electrode indirectly heats the contact means 170 via conduction, which is thereby brought above its melting temperature. The targeted heating therefore allows heating of the electrode 140 and the contact means 170 to a fixing temperature which is higher than the temperature of the regions of the catheter body 150 surrounding the contact opening 160. The surrounding regions of the catheter body are basically only raised directly adjacent to the electrode 140 and the contact means 170, but due to their low thermal conductivity, in particular the low thermal conductivity of polymeric inner liners 110 and outer insulation 130, there is no major thermal load for large parts of the catheter body 150. This can prevent adverse deformation of the catheter body 150.

FIG. 3f shows the final produced catheter 100, or a section thereof. The contact means 170 electrically conductively secures the electrode 140 to the electrical conductor 121, via its contact surface 122. In the shown embodiment of the method 200, securing is carried out by cooling the solder.

Additional electrodes 140 can be applied analogously to the described method 200, both distally as well as proximally, or at other desired positions. In so doing, individual method steps for all electrodes 140 can be carried out simultaneously or at least in concert before subsequent method steps are carried out. However, it may also be appropriate to attach each electrode 140 separately according to the described method 200.

### Reference Signs

100 catheter
110 inner liner
120 flexible circuit board
121 electrical conductor
122 contact surface
130 outer insulation
140 electrode
150 catheter body
160 contact opening
170 contact means
180 heating element
200 method for producing a catheter
210 providing a catheter body
220 creating a contact opening
230 providing a contact means
240 applying the contact means
250 providing an electrode
260 positioning the electrode
270 targeted heating

What is claimed is:

1. A method for producing a catheter, comprising the method steps of:
a) providing a catheter body, comprising a hollow cylindrical inner liner having an inner liner outer surface, the inner liner extending in a longitudinal direction from a proximal catheter body end to a distal catheter body end of the catheter body,
an outer insulation having an inner insulation surface, the outer insulation coaxially surrounding the inner liner,
a flexible circuit board which comprises at least one electrical conductor, wherein the flexible circuit board is arranged between the inner liner outer surface and the insulation inner surface, wherein the flexible circuit board is wound at least in sections in a spiral manner in turns around the inner liner, and wherein the inner liner and the outer insulation are integrally connected to one another in the spaces between the turns of the flexible circuit board;
b) creating at least one contact opening in the vicinity of the distal catheter body end, wherein, to create the contact opening, parts of the outer insulation and of any insulating layers of the flexible circuit board are removed so that the at least one electrical conductor can be selectively electrically contacted via the contact opening;
c) providing a contact means;
d) applying the contact means into the at least one contact opening;
e) providing at least one electrode;
f) positioning the electrode at the position of the contact opening in contact with the contact means in the contact opening;
g) targeted heating of the electrode and of the contact means in order to secure the electrode to the electrical conductor via the contact means in an electrically conductive manner.

2. The method according to claim 1, wherein the targeted heating in method step g) raises the electrode and the contact means to a fixing temperature which is higher than a temperature of the regions of the catheter body surrounding the contact opening.

3. The method according to claim 1, wherein the targeted heating in method step g) is carried out without convection.

4. The method according to claim 1, wherein the electrode consists of a metal.

5. The method according to claim 4, wherein the metal is selected from the group consisting of platinum, iridium, tantalum, palladium, titanium, iron, gold, molybdenum, niobium, tungsten, nickel, chromium, cobalt, steel, nitinol, and alloys comprising at least one of these metals.

6. The method according to claim 1, wherein the targeted heating in method step g) is carried out by means of targeted irradiation of the electrode with electromagnetic radiation.

7. The method according to claim 6, wherein the electromagnetic radiation is generated by means of a laser.

8. The method according to claim 6, wherein the laser is an IR laser.

9. The method according to claim 1, wherein the targeted heating in method step g) is carried out by means of induction.

10. The method according to claim 1, wherein the targeted heating in method step g) is carried out via direct contact of the electrode with a heating element.

11. The method according to claim 10, wherein the heating element is a soldering iron.

12. The method according to claim 1, wherein the contact means is a solder, an sintering paste, or a conductive paste.

13. The method according to claim 1, wherein the targeted heating in method step g) is carried out to a temperature in a range between 200°C and 280°C.

14. The method according to claim 1, wherein the targeted heating in method step g) is carried out to a temperature which lies below the melting temperature of the electrode.

15. The method according to claim 2, wherein the targeted heating in method step g) is carried out without convection.
